(19) United States
(12) Patent Application Publication
(10) Pub. No.: US 2025/0231178 A1
(43) Pub. Date: Jul. 17, 2025

(54) A BIOMARKER FOR DETERMINING ALZHEIMER'S DISEASE

(71) Applicant: UNIVERSITY OF EASTERN FINLAND, Kuopio (FI)

(72) Inventors: Tarja MALM, Siilinjarvi (FI); Rashid GINIATULLIN, Kuopio (FI); Ilpo KURONEN, Kuopio (FI); Ilkka FAGERLUND; Dilyara NURKHAMETOVA, Kuopio (FI)

(21) Appl. No.: 18/855,672

(22) PCT Filed: Apr. 11, 2023
(86) PCT No.: PCT/FI2023/050199

§ 371 Date: Oct. 10, 2024

(30) Foreign Application Priority Data
Apr. 12, 2022 (FI) 20225324

(51) Int. Cl. G01N 33/50

(52) U.S. Cl. CPC G01N 33/5091; G01N 2333/705; G01N 2800/2821

(57) ABSTRACT

The present disclosure relates to a method for determining a risk of development or risk of presence of Alzheimer's disease in a human subject comprising analyzing an activity of a PIEZO1 receptor. The disclosure also relates to a kit for determining a risk of development or risk of presence of Alzheimer's disease according to the present method. The disclosure also relates to an in vitro use of a PIEZO1 receptor as a biomarker and determination of intracellular calcium level as a biomarker for determining a risk of development or risk of presence of Alzheimer's disease in a human subject.

### FIELD OF THE DISCLOSURE

The present disclosure relates to a method for determining a risk of development or risk of presence of Alzheimer's disease in a human subject comprising analyzing an activity of a PIEZO1 receptor. The disclosure also relates to a kit for determining a risk of development or risk of presence of Alzheimer's disease according to the present method. The disclosure also relates to an in vitro use of a PIEZO1 receptor as a biomarker for determining a risk of development or risk of presence of Alzheimer's disease in a human subject. The disclosure also relates to an in vitro use of determining intracellular calcium level in response to the activation of PIEZO1 as a biomarker for determining a risk for development of Alzheimer's disease or risk for presence of Alzheimer's disease in a human subject.

### BACKGROUND OF THE DISCLOSURE

The earliest signs of the Alzheimer's disease (AD) include a Mild Cognitive Impairment (MCI), which is usually the first sign when symptoms give reason to suspect that the individual may have an AD. Commonly, this is the first time when the individual meets his/her doctor due to short-term memory loss, difficulty finding words, and/or losing time dependency on the normal routines. Other symptoms of MCI may include a decline in thinking, ability to make decisions or to complete tasks, which have previously been normal to the individual (Wei et al. 2021).

In many case, general practitioners or primary care providers are the first professionals to encounter with those problems. Diagnosis is made on a basis of a few core elements such as suspect's medical history including symptoms and past medical conditions and family history, questionnaire to assess the suspect's daily activities and compliance to the routines, and physical check-up and laboratory tests to assess level of reversible cognitive decline.

Additional tests are usually required and the appointment with the neurologist include variety of more specific analysis to support the diagnosis. Since AD shall affect more than 150 million people and will cost more than 2 trillion EUR by the end of 2050, a timely and accurate diagnosis is of utmost importance to reduce this enormous human and economic burden. Even though, there are no effective treatment for the disease, the current interventions may slow down the progression of the disease and improve disease management in general.

Diagnosis of the Alzheimer's disease at very early stage is complicated and is commonly based on assessment of the impairment of the cognition followed by analysis of fluid biomarkers such as tau and AB in cerebrospinal fluid (CSF), and imaging of brain (Wei et al. 2021). At very early stage, when impairment of the cognition is not clearly assessable and cannot be ruled as a progressive disease, the subjects being vulnerable of progressing to the active diseases may not be caught with the methods available. Also, intensive screening of the population is far too expensive with the imaging methods. The fluid biomarkers, on the other hand, may offer a feasible tool for efficient screening but their predictive value need to be better than that of tau or AB in serum.

As reported by Grothe et al. 2021, the recent guidelines of the National Institute on Aging-Alzheimer's Association (NIA-AA) Research Framework now define Alzheimer disease (AD) as a biological entity for which an in vivo diagnosis is no longer based solely on clinical diagnostic criteria but requires supporting evidence from PET or fluid biomarkers of AD-typical β-amyloid (Aβ) and tau pathology (Grothe et al. 2021). In contrast to PET, bodily fluid-based measurements can provide different molecular biomarkers from a single assessment and are more cost effective, widely attainable, and not limited by radiation exposure (Mattsson et al. 2009). The recent development of assays to measure phosphorylated tau in blood offers an alternative opportunity to assess AD pathology in a cost-effective, highly accessible, and scalable manner (Ashton et al. 2018; Scholl et al. 2019; Jack et al. 2018). Plasma concentrations of tau phosphorylated at threonine 181 (p-tau181) correlate highly with CSF measures of p-tau181 and with PET measures of AB and tau pathology and have been shown to distinguish between AD and other neurodegenerative disorders with high diagnostic accuracy comparable to that of CSF and PET-based measures of tau pathology (Grothe et al. 2021; Mattsson et al. 2009).

Fluid biomarkers such as Aβ and tau are well measurable in CSF (cerebrospinal fluid) with immunoassay (Ashton et al. 2018; Scholl et al. 2019; Jack et al. 2018). The results are used as an aid for diagnosis AD. These biomarkers indicate the AD pathology, which has been shown to relate to progressive AD. Fluid biomarkers such as tau and Aβ can be measured also in serum or plasma with lower reproducibility than using CSF as a sample. Therefore, accurate laboratory diagnostics of AD pathology prefers using CSF as a sample (Wei et al. 2021).

413 clinical trials aiming at development of new treatments for AD between 2022-2012 had a 99.6% failure rate (Cummings et al. 2014). One possible reason for this high failure rate has been misleading diagnostics with methods having poor diagnostic performance since subjects are often diagnosed with AD dementia, but they are found not to have AD pathology. Abner et al. previously reported that almost 10% to 30% of individuals clinically diagnosed as AD dementia did not display typical neuropathological manifestations at autopsy. Also, it is not possible to confirm the presence of amyloid pathology in cognitively normal subjects based on clinical presentation without biomarkers. Thus, AD biomarkers are vital tools in the inclusion criteria of clinical trials to confirm the presence of therapeutic target (Abner et al. 2017).

Another problem related to the currently known fluid biomarkers for AD relates to their prognostic value since they do not differentiate the AD suspects from the healthy at very early stage when the first signs such as MCI appear. Mild cognitive impairment (MCI) represents a stage where cognitive decline is greater than expected for one's age and education level but that does not interfere with activities of daily life. However, previous studies have shown that only an estimated 10% to 15% of MCI individuals will convert to dementia per year. While the application of biomarkers based on the NIAAA research framework enables the identification of MCI individuals due to AD, the clinical and biomarker characteristics of those who will progress to dementia within typical trial periods remain unclear (Petersen et al. 1985).

Patent publication WO2021/005268 discloses that a mechanosensitive receptor PIEZO1 is expressed in the brain in microglia cells. WO2021/005268 discloses a method of determining a neurodegenerative disease by detecting reduced PIEZO1 receptor activity using mouse blood cell as an experimental model. Glial cells maintain homeostasis and provide support and protection for neurons in brain. They are also regulators of the inflammation in brain and related to the pathological process in AD. Cahalan et al. 2015 and Wang et al. 2016 disclose that PIEZO1 is involved in regulation of blood pressure and red blood cell volume, respectively.

Despite the advances in the field of AD biomarkers there remains a need of improved methods and products, such as more sensitive and specific diagnostic.

### BRIEF DESCRIPTION OF THE DISCLOSURE

An object of the present disclosure is to provide a method, a kit as well as a use of a biomarker which overcome the above problems related to the presently used methods for diagnosing Alzheimer's disease.

The present disclosure relates to diagnosing or determining a risk of development or presence of Alzheimer's disease in a human subject by analyzing activity of a PIEZO1, a mechanosensitive calcium ion channel receptor in blood sample, such as peripheral human red blood cells (RBCs). Alzheimer's disease may be prodromal Alzheimer's disease or Alzheimer's disease with dementia. The method of the present disclosure comprises analyzing an activity of a PIEZO1 receptor. More specifically, the present disclosure relates to a method, in which the human peripheral blood, preferably human red blood cells, which have been withdrawn from the human body and placed into a receptacle and are treated with a PIEZO1 activator, preferably a PIEZO1 agonist such as Yoda1, Jedi1, or Jedi2. Activation of the PIEZO1 channel is measured for example with a fluorometry by using a fluorochrome, such as a calcium reactive fluorochromes, for example Fluo4 am or Fura2, which reacts with calcium and cause a measurable signal. Activity of the PIEZO1 in human blood cells, preferably red blood cells upon activation with the PIEZO1 specific agonists reflects the state of the Alzheimer's disease. The said method can be performed in a test tube with a fluorescent activated cell sorter, or in an advantageous embodiment, in a high throughput screening platform (HTS) comprising a microtiter plate with one or more wells and a fluorometer.

The present disclosure provides a method based on a surprising finding that increased response to the activation of PIEZO1 relates to increased risk for development of Alzheimer's disease or risk of presence of Alzheimer's disease in humans. The present disclosure discloses results from measurement of red blood cells from humans, including subjects diagnosed with Alzheimer's disease. Especially, the present disclosure provides a method of measuring a level of an intracellular calcium signal in response of the activation of PIEZO1 in an in vitro sample from a human subject. In the present method, higher intracellular calcium level, as detected for example using a fluorescent calcium indicator relates to high risk for development or presence of Alzheimer's disease in a subject. In other words, higher intracellular calcium level indicates high risk for development or presence of Alzheimer's disease in a subject.

Thus, the present disclosure provides a method for determining a risk of development of Alzheimer's disease or risk of presence of Alzheimer's disease in a human subject, wherein the method comprises the steps of:
a) providing an in vitro sample comprising blood cells from said human subject;
b) adding to the sample a reagent or reagents for detecting intracellular calcium in said sample;
c) measuring a baseline level of an intracellular calcium signal in said sample;
d) activating a PIEZO1 receptor in said sample to induce calcium influx;
e) measuring a level of an intracellular calcium signal in response of the activation of PIEZO1 in the sample;
f) calculating an increase in the level of the intracellular calcium signal from step c) to step e), wherein the increase in the level of the intracellular calcium signal from step b) to step e) provides a parameter, which parameter is used to determine the risk of development or risk of presence of Alzheimer's disease in said human subject,
   wherein increase in a value of the parameter obtained in step f) indicates an increased risk of development of Alzheimer's disease or increased risk of presence of Alzheimer's disease in said human subject.

The increase in a value of the parameter obtained in step f) may be directly proportional with the risk of development of Alzheimer's disease or presence of Alzheimer's disease in said human subject.

The present disclosure also provides a kit for determining a risk of development of Alzheimer's disease or risk of presence of Alzheimer's disease according to the presently described method, wherein the kit comprises a receptacle; an intracellular calcium indicator; a PIEZO1 activator; and optionally washing buffer.

The present disclosure also provides a use of a PIEZO1 receptor as a biomarker for determining a risk of development of Alzheimer's disease or risk of presence of Alzheimer's disease in a human subject. The PIEZO1 receptor is activated, said activation resulting in an increased level of an intracellular calcium signal thereby providing a parameter, which parameter is used to determine the risk of development or risk of presence of Alzheimer's disease in said human subject by comparing the obtained parameter to a known reference value for a subject with little or no risk of having Alzheimer's disease and/or to a known reference value for a subject with Alzheimer's disease to determine. The increase in a value of the obtained parameter may be directly proportional with the risk of development of Alzheimer's disease or presence of Alzheimer's disease in said human subject. Alternatively, the risk for development or presence of Alzheimer's disease may be calculated by comparing the obtained parameter to established probability density functions, based on a database of said parameters recorded from samples given human subjects with known health status regarding Alzheimer's disease. Preferably, the use is in vitro use.

The present disclosure also relates to an in vitro use of determining intracellular calcium level in response to the activation of PIEZO1 as a biomarker for determining a risk for development of Alzheimer's disease or risk for presence of Alzheimer's disease in a human subject

### BRIEF DESCRIPTION OF THE DRAWINGS

FIG. 1 shows one embodiment of the disclosure comprising the first step (1a) to transfer one or more blood samples into the microtiter plate having one or more wells (102) with pipette (101), and the second step (1b) to add a calcium reactive fluorochrome such as Fluo4 am, and the third step (1c) to add a PIEZO1 agonist such as Yoda1, Jedi1, or Jedi2, and the fourth step (1d) to read the result with a fluorometer (103), and interpret the results by comparing the obtained parameter to a known reference value for a subject with little or no risk of having Alzheimer's disease and/or to a known reference value for a subject with Alzheimer's disease. An example of results comparing subjects with little or no risk of having AD and subjects with prodomal AD (MCI) and AD with dementia (AD) are shown in FIGS. 3A-3F. The steps 1a-1d can be automated to speed up the process.

FIG. 2 shows one embodiment of the disclosure comprising the first step (2a) to transfer a blood sample into the test tube (108) with a pipette (101), and the second step (2b) to add a calcium reactive fluorochrome such as Fluo4 am, and the third step (2c) to add a PIEZO1 agonist such as Yoda1, Jedi1, or Jedi2, and the fourth step (2d) to read the result with a fluorescence activated cell sorter (FACS) (109), and interpret the results by comparing the obtained parameter to a known reference value for a subject with little or no risk of having Alzheimer's disease and/or to a known reference value for a subject with Alzheimer's disease. Example of results comparing subjects with little or no risk of having AD and subjects with AD are shown in FIG. 3G.

FIGS. 3A-3C show Yoda1-induced (10 μM) Ca-flux in red blood cells (RBCs) from control subjects, patients with MCI, and AD. The time course of Fluo4 am fluorescence intensity in response to application of Yoda1 10 μM. First time point represents baseline value (BL), Yoda1 is applied at 20 s of recording. Median fluorescence values (MFI values) are normalized on BL.

FIG. 3A shows Yoda1-induced response in red blood cells from control subjects (n=23, white circles) and in red blood cells from subjects with mild cognitive impairment (MCI) (n=26, black squares).

FIG. 3B shows Yoda1-induced response in red blood cells from control subjects (n=23, white circles) and in red blood cells from AD patients (n=21, black squares).

FIG. 3C shows Yoda1-induced response in red blood cells from subjects with MCI (n=26, white circles) and in red blood cells from AD patients (n=21, black squares). Mean±SEM.

FIGS. 3D-3F show histograms comparing response to Yoda-1 at 60 s time point. Mann-Whitney test, two-tailed, ****p<0.0001

FIG. 3D shows Yoda1-induced response in blood cells from control subjects (white bars, n=23) and in red blood cells from patients with MCI (light grey, n=26).

FIG. 3E shows Yoda1-induced response in red blood cells from control subjects (white bars, n=23) and in red blood cells from AD patients (dark grey, n=21).

FIG. 3F shows Yoda1-induced response in in red blood cells from patients with MCI (light grey, n=26) and in red blood cells from AD patients (dark grey, n=21). Mean±SEM, Mann-Whitney test, two-tailed, ****p<0.0001.

FIG. 3G shows Yoda1-induced (5 μM) Ca-flux in red blood cells (RBCs) in control subjects (n=4, white circles) and patients with AD (n=4, black squares) on the microtitre plate and measured on a plate reader. Mann-Whitney test, two-tailed, *p<0.05. The Y-axis shows the fluorescence signal after addition of Yoda1, normalised to the baseline recorded before addition of Yoda1. Signals recorded without addition of Fluo4, with and without Yoda1, were subtracted from their Fluo4 loaded counterparts to give the final read-outs used in normalising the results.

### DETAILED DESCRIPTION OF THE DISCLOSURE

The present disclosure provides a method for determining a risk of development or risk of presence of Alzheimer's disease in a human subject, wherein the method comprises the steps of:
a) providing an in vitro sample comprising blood cells from said human subject;
b) adding to the sample a reagent or reagents for detecting intracellular calcium in said sample;
c) measuring a baseline level of an intracellular calcium signal in said sample;
d) activating a PIEZO1 receptor in said sample to induce calcium influx;
e) measuring a level of an intracellular calcium signal in response of the activation of PIEZO1 in the sample;
f) calculating an increase in the level of the intracellular calcium signal from step c) to step e), wherein the increase in the level of the intracellular calcium signal from step c) to step e) provides a parameter, which parameter is used to determine the risk of development or risk of presence of Alzheimer's disease in said human subject,
   wherein increase in a value of the parameter obtained in step f) indicates an increased risk of development of Alzheimer's disease or increased risk of presence of Alzheimer's disease in said human subject.

Term “biomarker” or “biological marker” refers to a medical sign which can be measured accurately and reproducibly. The biomarker may be an objective indication of medical state observed from outside a patient. Biomarker may be a substance, structure, or process or method that can be measured in the body or its products and influence or predict the incidence of outcome or disease.

As used herein in the present disclosure a PIEZO1 receptor is used as a biomarker, which can be used to assess the subjects who will most likely develop Alzheimer's disease (AD).

“PIEZO1” is a mechanosensitive ion channel protein that in humans is encoded by the gene PIEZO1. The term “PIEZO1 receptor” refers to PIEZO1. Structure and function of PIEZO1 is presented for example by Jiang et al. 2021 and Fang et al. 2021. PIEZO1 is referred to as HGNC gene id 28993 and uniProt entry number Q92508.

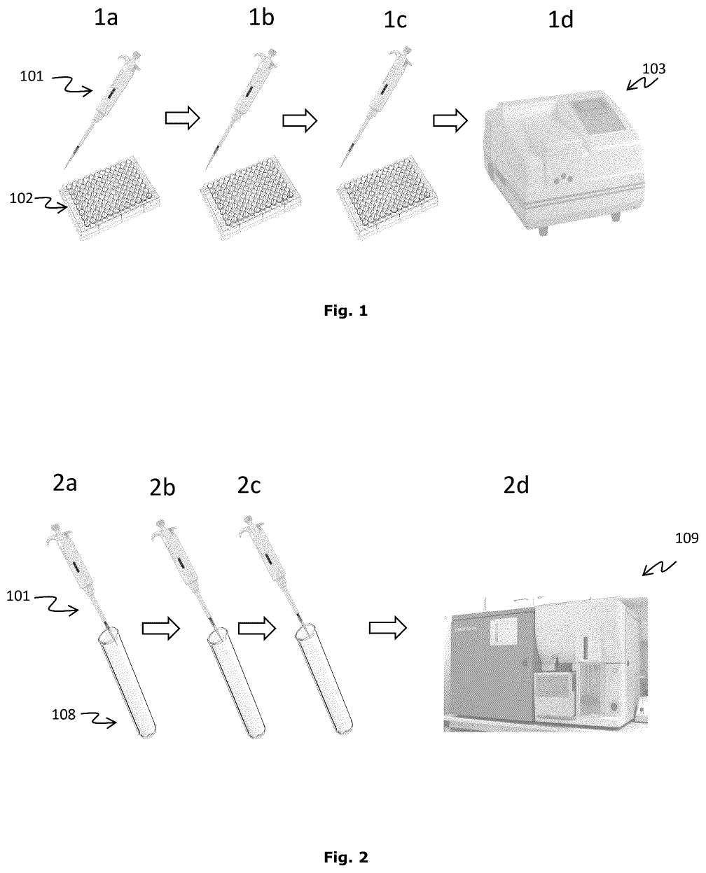

PIEZO1 is a mechanosensitive receptor, which responds to mechanical forces such as shearing and pressure on cell membranes. The response is mediated into the cell by an Ca²⁺ efflux, which increases Ca²⁺ concentration in the cytoplasm. This phenomenon makes it possible to measure PIEZO1 gating properties with the Ca²⁺ ion induced fluorochromes. Binding of the Ca²⁺ ions to the calcium induced fluorochrome, induces fluorescence, which can be measured with the fluorometers. The methods, which are applicable to measure PIEZO1 gating activity by means of Ca²⁺ induced fluorescence include fluorescence activated cell sorter (FACS) (FIG. 2), and in a more advantageous embodiment, a high through-put screening (HTS) method using a microtitration plate with one or more wells (FIG. 1). The HTS method enables to measure large number of samples in a short time with automation.

In use as a biomarker PIEZO1 receptor is activated, which activation results in an increased level of an intracellular calcium signal. An increase from the baseline level of the intracellular calcium signal in the sample to the level of the intracellular calcium signal in response of the activation of PIEZO1 in the sample is calculated. The increased level of the intracellular calcium signal provides a parameter, which is used to determine the risk of development or risk of presence of Alzheimer's disease in said human subject. The obtained parameter is compared to a known reference value for a subject with little or no risk of having Alzheimer's disease and/or to a known reference value for a subject with Alzheimer's disease to determine a risk of development or risk of presence of Alzheimer's disease in a human subject. Alzheimer's disease may be prodromal Alzheimer's disease or Alzheimer's disease with dementia.

In an embodiment, the risk for development or presence of Alzheimer's disease may be calculated by comparing the obtained parameter to established probability density functions, based on a database of said parameters recorded from samples given human subjects with known health status regarding Alzheimer's disease.

Analysis of the biomarker in the present disclosure is especially useful for early detection of AD subjects. As used herein, the biomarker refers to a PIEZO1 receptor found in many different cells in human body, and more specifically, the biomarker refers to the activity of the PIEZO1 receptor in RBCs as a function of its specific agonists such as Yoda1, Jedi1, or Jedi2. It regulates many vital functions in human and its dysfunction is shown to be related to the Aβ pathology and neuroinflammation in brain. As used herein, “in blood” means that the biomarker exists in RBCs and can be detected in blood specimen.

The term “a subject with Alzheimer's disease” or “a human subject with Alzheimer's disease” or “a subject having Alzheimer's disease” refers to a patient, individual or subject who is diagnosed with Alzheimer's disease by a qualified physician. Alzheimer's disease (AD) may be prodromal Alzheimer's disease (AD) or Alzheimer's disease (AD) with dementia.

“Mild Cognitive Impairment” or “MCI” is usually the first sign when symptoms give reason to suspect that the individual may have an AD. MCI is a neurocognitive disorder which involves cognitive impairments beyond those expected based on an individual's age and education but which are not significant enough to interfere with instrumental activities of daily living. MCI may occur as a transitional stage between normal aging and dementia, especially Alzheimer's disease.

The term “healthy subject” as regards with Alzheimer's disease refers to a patient, individual or subject with little or no risk of having Alzheimer's disease.

It is known that peripheral red blood cells (RBC) express PIEZO1 receptors, which maintain many vital functions of RBCs. The present inventors surprisingly found that activity of the PIEZO1 receptor in RBCs after addition of a PIEZO1 specific agonist, such as Yoda1, Jedi1, or Jedi2, is different in the samples collected from the healthy subjects i.e., subjects with little or no risk of having Alzheimer's disease and from the AD patients. Similar difference was evident when comparing samples from healthy subjects to samples from donors diagnosed with MCI due to Alzheimer's disease, i.e., prodromal Alzheimer's disease, justifying the biomarker's utility in detecting the presence of MCI without prior knowledge of the donor's cognitive state.

The present disclosure includes an in vitro method of measuring intracellular calcium from human red blood cells, including blood cells from subjects diagnosed with Alzheimer's disease.

Especially, the present disclosure provides a method of measuring a level of an intracellular calcium signal in response of the activation of PIEZO1 in a human sample. In the present method, higher intracellular calcium level, as detected for example using a fluorescent calcium indicator, relates to high risk for development of Alzheimer's disease or presence of Alzheimer's disease in a subject. In other words, higher intracellular calcium level indicates high risk for development or presence of Alzheimer's disease in a subject. In preferred embodiments of the present method the in vitro measurements are carried out from red blood cells from humans.

The present disclosure provides a surprising finding that increased response to the activation of PIEZO1 relates to increased risk for development of Alzheimer's disease or risk of presence of Alzheimer's disease in humans. The present disclosure discloses results from measurement of red blood cells from humans, including subjects diagnosed with Alzheimer's disease.

The higher the increase in the intracellular calcium signal is the higher the risk of development of Alzheimer's disease or risk of presence of Alzheimer's disease in said human subject. In an embodiment, the risk for development or presence of Alzheimer's disease may be calculated by comparing the obtained parameter to established probability density functions, based on a database of said parameters recorded from samples given human subjects with known health status regarding Alzheimer's disease. A skilled person is in the art is aware of suitable probability density functions as well as suitable databases to be used.

The present inventors show connection between the response to the activation of PIEZO1, for example to the activation with a PIEZO1 agonist, such as Yoda1, Jedi1, or Jedi2, and cognitive impairment (prodromal AD and AD with dementia) compared to the healthy (normal cognition) in RBCs. Increased response to the activation of PIEZO1, such as the response to the activation with for example Yoda1 means that the subject has an abnormal PIEZO1 function, which may indicate progressive neurological disorder leading to the development of AD.

The disclosure describes a method for determining a risk of development or risk of presence of Alzheimer's disease in a human subject. The method comprises analyzing an activity of a PIEZO1 receptor.

Especially the method of the present disclosure is based on measuring and calculating an increase of the intracellular calcium signal from the baseline level of a sample to a level in response to the activation of PIEZO1 in the sample. The measured increase of the intracellular calcium signal provides a parameter, which parameter is used to determine the risk of development or risk of presence of Alzheimer's disease in said human subject.

The present disclosure relates to use of PIEZO1 receptor as a biomarker in test methods for AD. The present method is useful for early diagnosis of AD, and differentiation of the MCI and AD subjects from the healthy subjects i.e., persons who have no measurable impairment in cognition. Since about 10-30% of the MCI subjects develop AD within one year of MCI diagnosis (Ward et al. 2013), the test method can be used on an objective basis for a risk assessment of a person to develop the disease. The method can be used by itself or as an auxiliary means of diagnosing Alzheimer's disease, whether characterized by MCI or dementia.

The method of the present disclosure is characterized by that it uses addition of the calcium (Ca²⁺) inducible or calcium reactive fluorochromes, such as Fluo4 am or Fura2, and activation of the PIEZO1 receptor in blood cells, such as RBCs, with PIEZO1 specific agonists, such as Yoda1, Jedi1, or Jedi2, to produce a signal based on which the method can differentiate healthy subjects from subjects with MCI or AD.

In the test method of the present disclosure, the biomarker can be measured using blood as a sample.

Thus, the present disclosure provides a method for determining a risk of development or risk of presence of Alzheimer's disease in a human subject, wherein the method comprises the steps of:
a) providing an in vitro sample comprising blood cells from said human subject;
b) adding to the sample a reagent or reagents for detecting intracellular calcium in said sample;
c) measuring a baseline level of an intracellular calcium signal in said sample;
d) activating a PIEZO1 receptor in said sample to induce calcium influx;
e) measuring a level of an intracellular calcium signal in response of the activation of PIEZO1 in the sample;
f) calculating an increase in the level of the intracellular calcium signal from step c) to step e), wherein the increase in the level of the intracellular calcium signal from step c) to step e) provides a parameter, which parameter is used to determine the risk of development or risk of presence of Alzheimer's disease in said human subject,
   wherein increase in a value of the parameter obtained in step f) indicates an increased risk of development of Alzheimer's disease or increased risk of presence of Alzheimer's disease in said human subject.

In an embodiment Alzheimer's disease is prodromal Alzheimer's disease or Alzheimer's disease with dementia.

In an embodiment the method further comprises a step of washing the blood cells with a physiological buffer and centrifugation after step a). The physiological buffer may for example be phosphate-buffered saline (PBS) or Hank's Balanced Salt Solution (HBSS). A skilled person is able to select a suitable buffer among the physiological buffers.

In an embodiment the method comprises calculating an increase from the baseline level of the intracellular calcium signal in the sample to the level of the intracellular calcium signal in response of the activation PIEZO1 in the sample, wherein the increased level of the intracellular calcium signal provides a parameter, which parameter is used to determine the risk of development or risk of presence of Alzheimer's disease in said human subject. Increased level of the intracellular calcium signal may also be referred as an elevated level of the intracellular calcium signal.

In an embodiment the method comprises an in vitro method of determining a risk of development or risk of presence of Alzheimer's in a human subject, wherein the method comprises the steps of:
a) providing an in vitro sample comprising blood cells from said human subject;
b) adding to the sample a reagent or reagents for detecting intracellular calcium in said sample;
c) measuring a baseline level of an intracellular calcium signal in said sample;
d) activating a PIEZO1 receptor present in said sample to induce calcium influx;
e) measuring a level of an intracellular calcium signal in response of the activation of PIEZO1 in the sample;
f) calculating an increase in the level of the intracellular calcium signal from step c) to step e), wherein the increase in the level of the intracellular calcium signal from step c) to step e) provides a parameter, which parameter is used to determine the risk of development or risk of presence of Alzheimer's disease in said human subject,
   wherein increase in a value of the parameter obtained in step f) indicates an increased risk of development of Alzheimer's disease or increased risk of presence of Alzheimer's disease in said human subject.

The increase in a value of the parameter obtained in step f) may be directly proportional with the risk of development of Alzheimer's disease or presence of Alzheimer's disease in said human subject. In other words, increased response to the activation of PIEZO1 indicates an increased risk of development of Alzheimer's disease or presence of Alzheimer's disease in said human subject.

In an embodiment, increased response to the activation of PIEZO1 indicates an increased risk of developing prodromal Alzheimer's disease or Alzheimer's disease with dementia, or increased risk of presence of prodromal Alzheimer's disease or Alzheimer's disease with dementia.

In an embodiment, increased response to the activation of PIEZO1 compared to healthy subjects indicates an increased risk of developing or presence of prodromal Alzheimer's disease or Alzheimer's disease with dementia.

In an embodiment the method comprises an in vitro method for determining a risk of development or risk of presence of prodromal Alzheimer's disease or Alzheimer's disease with dementia in a human subject, wherein the method comprises the steps of:
a) providing an in vitro sample comprising blood cells from said human subject;
b) adding to the sample a reagent or reagents for detecting intracellular calcium in said sample;
c) measuring a baseline level of an intracellular calcium signal in said sample;
d) activating a PIEZO1 receptor present in said sample to induce calcium influx;
e) measuring a level of an intracellular calcium signal in response of the activation of PIEZO1 in the sample;
f) calculating an increase in the level of the intracellular calcium signal from step c) to step e), wherein the increase in the level of the intracellular calcium signal from step c) to step e) provides a parameter, which parameter is used to determine the risk of development or risk of presence of Alzheimer's disease in said human subject,
   wherein increase in a value of the parameter obtained in step f) indicates an increased risk of development of Alzheimer's disease or increased risk of presence of Alzheimer's disease in said human subject.

In an embodiment the method further comprises comparing the obtained parameter to a known reference value for a subject with little or no risk of having Alzheimer's disease and/or comparing the obtained parameter to a known reference value for a subject having Alzheimer's disease to determine a risk of development or risk of presence of Alzheimer's disease in a human subject.

In an embodiment the method comprises an in vitro method for determining a risk of development or risk of presence of prodromal Alzheimer's disease or Alzheimer's disease with dementia in a human subject, wherein the method comprises the steps of:
a) providing an in vitro sample comprising blood cells from said human subject;
b) adding to the sample a reagent or reagents for detecting intracellular calcium in said sample;
c) measuring a baseline level of an intracellular calcium signal in said sample;
d) activating a PIEZO1 receptor present in said sample to induce calcium influx;
e) measuring a level of an intracellular calcium signal in response of the activation of PIEZO1 in the sample;
f) calculating an increase in the level of the intracellular calcium signal from step c) to step e), wherein the increase in the level of the intracellular calcium signal from step c) to step e) provides a parameter, which parameter is used to determine the risk of development or risk of presence of Alzheimer's disease in said human subject;
g) comparing the obtained parameter to a known reference value for a subject with little or no risk of having Alzheimer's disease and/or to a known reference value for a subject with Alzheimer's disease to determine a risk of development or risk of presence of Alzheimer's disease in a human subject;
   wherein increased response to the activation of PIEZO1 compared to healthy subjects indicates an increased risk of developing or presence of prodromal Alzheimer's disease or Alzheimer's disease with dementia.

In an embodiment a read-out from the human subject's sample is analyzed against a probability distribution, assigning for each read-out a probability of being identified as healthy, a probability for being identified as having prodromal Alzheimer's disease and a probability for being identified having Alzheimer's disease with dementia, wherein the probability distributions are based on previously analyzed cohorts of donors for each respective subject group.

In an embodiment the method is an in vitro method.

The present method for determining a risk of development or risk of presence of Alzheimer's disease in a human subject comprises the following step: providing a sample comprising blood cells from said human subject.

In an embodiment the sample comprises red blood cells (RBCs).

The present method for determining a risk of development or risk of presence of Alzheimer's disease in a human subject also comprises the following step: adding to the sample a reagent or reagents for detecting intracellular calcium in said sample.

In an embodiment the reagent or reagents for detecting intracellular calcium comprises a fluorochrome. In a preferred embodiment of the disclosure, the fluorochrome is a calcium reactive fluorochrome. The calcium reactive fluorochrome reacts with calcium and gives a signal measurable with a fluorometer. An example of a fluorochrome is Fluo4 am.

In another embodiment the reagent or reagents for detecting intracellular calcium comprise luminescent agent, such as coelenterazine and its analogs.

A skilled person can apply suitable fluorescent or luminometric detectors in the present method. Examples of suitable calcium indicators include such as synthetic Ca²⁺ dyes Fluo-4, Fluo-5F, Fluo-4FF, Rhod-2, X-Rhod-5F, Oregon Green 488 BAPTA-6F, Fluo-8, Fluo-8 high affinity, Fluo-8 low affinity, Oregon Green BAPTA-1, Cal-520, Rhod-4, Asante Calcium Red, and X-Rhod-1 as well as genetically encoded Ca²⁺-indicators such as GCaMP6-slow, -medium and -fast variants can be used in the invention. Preferably, the indicator is Fluo-4 or Fura2.

The intracellular calcium signal may be measured using fluorescence or luminescence. Typically, fluorescence is measured using a fluorometer or fluorescence activated cell sorter (FACS) (FIG. 2). Typically, luminescence is measured using a luminometer. Typically, microtiter plate readers (FIG. 1) can measure both fluorescence and luminescence.

The present method for determining a risk of development or risk of presence of Alzheimer's disease in a human subject also comprises the following step: measuring a baseline level of an intracellular calcium signal in said sample.

The term “baseline level” refers to the signal measured before activation. “Baseline level of the intracellular calcium signal” refers to the signal before activation of PIEZO1.

The present method for determining a risk of development or risk of presence of Alzheimer's disease in a human subject also comprises the following step: activating a PIEZO1 receptor in the sample to induce calcium influx.

In an embodiment of the present disclosure the PIEZO1 receptor is activated by a PIEZO1 agonist. In a preferred embodiment the PIEZO1 agonist is Yoda1, Jedi1, or Jedi2.

The term “agonist” refers to those agents that can, by binding to a macromolecule target, positively influence the process, e.g., can activate or stimulate the process, and include chemical, biochemical, cellular or physiological processes. It includes but is not limited to these. In other words, an agonist is a chemical that activates a receptor to produce a biological response.

In the present disclosure the term “agonist” refers to a chemical that can induce increases in Ca influx through modulating the function of PIEZO1. Examples of PIEZO1 modulation include but, are not limited to, lowering the threshold of activation, and altering the activation and inactivation (i.e., opening and closing of the channel) kinetics of PIEZO1.

The term “PIEZO1 agonist” refers to a molecule capable of activating a PIEZO1 receptor. A piezo agonist can bind to a PIEZO1 receptor. Examples of a PIEZO1 agonists are Yoda1 (Sveda et al. 2015), Jedi1, and Jedi2 (Wang Y. et al. 2018).

In an embodiment the PIEZO1 agonist is 2-[(2,6-Dichlorophenyl)methylsulfanyl]-5-pyrazin-2-yl-1,3,4-thiadiazole, referred to as Yoda1. In an another embodiment the PIEZO1 agonist is 2-methyl-5-phenylfuran-3-carboxylic acid, referred to as Jedi1. In another embodiment the PIEZO1 agonist is 2-Methyl-5-(thien-2-yl)-3-furoic acid, referred to as Jedi2.

Formulae of the compounds below have been drawn using OpenBabelGUI (version 2.4.0, http://openbabel.org) (O'Boyle et al. 2011) and respective reference mentioned at each compound.

Yoda1 (2-[(2,6-Dichlorophenyl)methylsulfanyl]-5-pyrazin-2-yl-1,3,4-thiadiazole) has the formula I (National Center for Biotechnology Information (2022). PubChem Compound Summary for CID 2746822. Retrieved Apr. 5, 2022 from https://pubchem.ncbi.nlm.nih.gov/compound/Yoda-1)

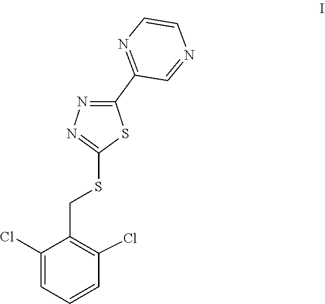 [SMILES: Clc1cccc(Cl)c1CSc1nnc(-c2cnccn2)s1]

Jedi1 (2-methyl-5-phenylfuran-3-carboxylic acid) has the formula II (National Center for Biotechnology Information (2022). PubChem Substance Record for SID 381744931, Jedi1, Source: IUPHAR/BPS Guide to PHARMACOLOGY. Retrieved Apr. 5, 2022 from https://pubchem.ncbi.nlm.nih.gov/substance/381744931)

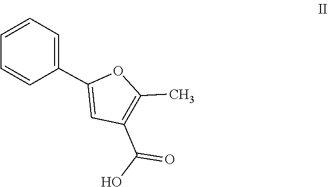

Jedi2 (2-Methyl-5-(thien-2-yl)-3-furoic acid) has the formula III (National Center for Biotechnology Information (2022). PubChem Compound Summary for CID 2796026, 2-Methyl-5-(thien-2-yl)-3-furoic acid. Retrieved from Apr. 5, 2022 https://pubchem.ncbi.nlm.nih.gov/compound/2-Methyl-5-thien-2-yl-3-furoic acid)

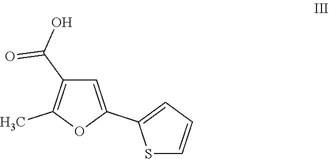

The amount of PIEZO1 agonist may between 1 nM-50 μM. Preferably, the amount can be 100 nM, 200 nM, 300 nM, 400 nM, 500 nM, 600 nM, 700 nM, 800 nM, 900 nM, 100 nm-1 μM, 500 nM-1 μM, 1 μM-25 μM, 1 μM-20 μM, 1 μM-15 μM, 1 μM-10 μM, 1 μM-5 μM, 1 μM-50 μM, 5 μM-10 μM, 5 μM-15 μM, 10 μM-15 μM, 10 μM-20 μM, 10 μM-25 μM, 10 μM-30 μM, 15 μM-30 μM, 20 μM-30 μM, 25 μM-30 μM, 30 μM-40 μM, 30 μM-50 μM, 40 μM-50 μM, 45M-50 μM, 1 μM, 2 μM, 3 μM, 4 μM, 5 μM, 6 μM, 7 μM, 8 μM, 9 μM, 10μ, 11μ, 12μ, 13μ, 14μ, 15μ, 16μ, 17μ, 18μ, 19μ, 20μ M, 21 μM, 22 μM, 23 μM, 24 μM, 25 μM, 26 μM, 27 μM, 28 μM, 29 μM, 30 μM, 40 μM or 50M. More preferably, the amount is 1 μM-50 μM.

In an embodiment the above specified amounts concern Yoda1. In another embodiment the above specified amounts concern Jedi1. In another embodiment the above specified amounts concern Jedi2.

An “effective amount” is an amount of the compound(s) as described herein that induces on the expression and/or abundance of the ion channel or induces the activity of the ion channel.

PIEZO1 is a mechanosensitive receptor, which responds to mechanical forces such as shearing and pressure on cell membranes. Thus, the PIEZO1 receptor may be activated by mechanical intervention, such as change in osmotic pressure, ultrasound, shaking or induced convection in the sample fluid that mechanically activates PIEZO1.

The present method for determining a risk of development or risk of presence of Alzheimer's disease in a human subject also comprises the following step: measuring a level of an intracellular calcium signal in response of the activation of PIEZO1 in the sample.

The present method for determining a risk of development or risk of presence of Alzheimer's disease in a human subject also comprises the following step: calculating an increase in the level of the intracellular calcium signal from step c) to step e), wherein the increase in the level of the intracellular calcium signal from step c) to step e) provides a parameter, which parameter is used to determine the risk of development or risk of presence of Alzheimer's disease in said human subject.

Typical normalized increases in intracellular Ca²⁺ signal are in the range of 200%-2000%, where 100% is the baseline before activation of PIEZO1. However, depending on measurement set-up and the sample, the increase may be less than 200% or more than 2000%.

For example, the increase in intracellular Ca²⁺ signal may be in the range of 200-2000%, such as 200%, 300%, 400%, 500%, 600%, 700%, 800%, 900%, 1000%, 1100%, 1200%, 1300%, 1400%, 1500%, 1600%, 1700%, 1800%, 1900%, 2000% or in the range defined by any two of these values. In the present disclosure a healthy subject means a subject with little or no risk of having Alzheimer's disease.

Alzheimer's disease may be symptomatic or prodromal stage of Alzheimer's disease. Alzheimer's disease may be prodromal Alzheimer's disease or Alzheimer's disease with dementia.

In an embodiment, an “increase of a level” or “increase in the level” may be a difference between the level of intracellular Ca in response to PIEZO1 activation in a sample as compared to a control of at least about 1%, at least about 2%, at least about 3%, at least about 5%, at least about 10%, at least about 15%, at least about 20%, at least about 25%, at least about 30%, at least about 35%, at least about 40%, at least about 50%, at least about 60%, at least about 75%, at least about 80% or more. In one embodiment, an “increase of a level” or “increase in the level” may be a statistically significant difference between the level of intracellular Ca in response to PIEZO1 activation in a sample as compared to a control. For example, a difference may be statistically significant if the measured level of the biomarker falls outside of about 1.0 standard deviation, about 1.5 standard deviations, about 2.0 standard deviations, or about 2.5 stand deviations of the mean of any control or reference group. The reference or control can be, for example, a sample from a healthy individual, or a sample taken from the same individual at an earlier time point, such as a time point prior to administration of a therapeutic or an earlier time point during a therapeutic regimen.

In an embodiment the determining step f) comprises calculating an increase from the baseline level of the intracellular calcium signal in the sample to the level of the intracellular calcium signal in response of the activation PIEZO1 in the sample, wherein the increased level of the intracellular calcium signal provides a parameter, which parameter is used to determine the risk of development or risk of presence of Alzheimer's disease in said human subject.

In an embodiment, the determining step f) of the present method further comprises comparing the obtained parameter to a known reference value for a subject with little or no risk of having Alzheimer's disease and/or to a known reference value for a subject with Alzheimer's disease to determine a risk of development or risk of presence of Alzheimer's disease in a human subject.

In an embodiment a reference value or reference values indicating the absence of Alzheimer's disease in a subject or subjects have been previously established using blood samples from a cohort of healthy donors.

In an embodiment a reference value or reference values indicating prodromal Alzheimer's disease in a subject or subjects have been previously established using blood samples from a cohort of donors with diagnosis for Alzheimer's disease and diagnosed cognitive status of mild cognitive impairment (MCI).

In an embodiment a reference value or reference values indicating Alzheimer's disease in a subject or subjects have been previously established using blood samples from a cohort of donors with diagnosis for Alzheimer's disease and diagnosed with cognitive decline more severe than MCI.

In an embodiment reference value or reference values indicating prodromal Alzheimer's disease and Alzheimer's disease are higher than the reference values indicating healthy stage. The healthy stage means little or no risk of having Alzheimer's disease.

The absolute values of the reference values may be specific to the method of detecting and measuring intracellular calcium levels.

The term “normal result” refers a result, which is within the statistical range of the typical signals received with the samples collected from the healthy subjects, i.e. the subjects with little or no risk of having AD. This result is not an indication of progressive neurological process.

The term “abnormal result” refers to a result, which is within the statistical range of the typical signals received with the samples collected from the Alzheimer's patients. This result indicates that the subject has a neurological process typical to Alzheimer's disease for both prodromal Alzheimer's disease and Alzheimer's disease with dementia.

In an embodiment, a read-out from subject's may be analyzed against a probability distribution, assigning each read-out a probability of being identified as healthy, a probability for being identified as having prodromal Alzheimer's disease and a probability for being identified as having Alzheimer's disease with dementia. The probability distributions are based on previously analyzed cohorts of donors for each respective subject group.

In an embodiment of the present disclosure the relationship between the cognitive impairment and the PIEZO1 activity in the RBCs is dependent on activation with PIEZO1 specific agonist such as Yoda1, Jedi1, or Jedi2. Thus, diagnostic kits harboring reagent(s), which activates PIEZO1 receptor and makes the activation measurable with a fluorochrome or fluorochromes, preferably calcium reactive fluorochrome, such as Fluo4 am, can be developed and manufactured.

In an embodiment the present method is used for differentiating the healthy subjects and subjects with mild cognitive impairment.

In an embodiment the present method is used for differentiating the healthy subjects and subjects with Alzheimer's disease.

In an embodiment a method for determining a risk of development or risk of presence of Alzheimer's disease in a human subject comprises the steps of:
a) providing an in vitro sample comprising blood cells from said human subject;
b) optionally washing the blood cells with physiological buffer and centrifugation;
c) adding to the sample a reagent or reagents for detecting intracellular calcium in said sample;
d) measuring a baseline level of an intracellular calcium signal in said sample;
e) activating a PIEZO1 receptor in said sample to induce calcium influx;
f) measuring a level of an intracellular calcium signal in response of the activation of PIEZO1 in the sample;
g) calculating an increase in the level of the intracellular calcium signal from step c) to step e), wherein the increase in the level of the intracellular calcium signal from step c) to step e) provides a parameter, which parameter is used to determine the risk of development or risk of presence of Alzheimer's disease in said human subject,
   wherein increase in a value of the parameter obtained in step f) indicates an increased risk of development of Alzheimer's disease or increased risk of presence of Alzheimer's disease in said human subject.

In an embodiment a method for determining a risk of development or risk of presence of Alzheimer's disease in a human subject comprises the steps of:
a) providing an in vitro sample comprising blood cells from said human subject;
b) washing the blood cells with physiological buffer and centrifugation;
c) adding to the sample a reagent or reagents for detecting intracellular calcium in said sample;
d) measuring a baseline level of an intracellular calcium signal in said sample;
e) activating a PIEZO1 receptor in said sample to induce calcium influx;
f) measuring a level of an intracellular calcium signal in response of the activation of PIEZO1 in the sample;
g) calculating an increase in the level of the intracellular calcium signal from step c) to step e), wherein the increase in the level of the intracellular calcium signal from step c) to step e) provides a parameter, which parameter is used to determine the risk of development or risk of presence of Alzheimer's disease in said human subject,
   wherein increase in a value of the parameter obtained in step f) indicates an increased risk of development of Alzheimer's disease or increased risk of presence of Alzheimer's disease in said human subject.

In an embodiment a method for determining a risk of development or risk of presence of Alzheimer's disease in a human subject comprises the steps of:
a) providing an in vitro sample comprising blood cells from said human subject;
b) adding to the sample a reagent or reagents for detecting intracellular calcium in said sample;
c) measuring a baseline level of an intracellular calcium signal in said sample;
d) activating a PIEZO1 receptor in said sample to induce calcium influx;
e) measuring a level of an intracellular calcium signal in response of the activation of PIEZO1 in the sample;
f) calculating an increase from the baseline level of the intracellular calcium signal in the sample to the level of the intracellular calcium signal in response of the activation PIEZO1 in the sample, wherein the increased level of the intracellular calcium signal provides a parameter, which parameter is used to determine the risk of development or risk of presence of Alzheimer's disease in said human subject,
   wherein increase in a value of the parameter obtained in step f) indicates an increased risk of development of Alzheimer's disease or increased risk of presence of Alzheimer's disease in said human subject.

In an embodiment a method for determining a risk of development or risk of presence of Alzheimer's disease in a human subject comprises the steps of:
a) providing a sample comprising blood cells from said human subject;
b) optionally washing the blood cells with physiological buffer and centrifugation;
c) adding to the sample a reagent or reagents for detecting intracellular calcium in said sample;
d) measuring a baseline level of an intracellular calcium signal in said sample;
e) activating a PIEZO1 receptor in said sample to induce calcium influx;
f) measuring a level of an intracellular calcium signal in response of the activation of PIEZO1 in the sample;
g) calculating an increase from the baseline level of the intracellular calcium signal in the sample to the level of the intracellular calcium signal in response of the activation PIEZO1 in the sample, wherein the increased level of the intracellular calcium signal provides a parameter, which parameter is used to determine the risk of development or risk of presence of Alzheimer's disease in said human subject,
h) comparing the obtained parameter to a known reference value for a subject with little or no risk of having Alzheimer's disease and/or to a known reference value for a subject with Alzheimer's disease to determine,
   wherein the Alzheimer' disease is prodromal Alzheimer' disease or Alzheimer' disease with dementia.

In an embodiment a method for determining a risk of development or risk of presence of Alzheimer's disease in a human subject comprises the steps of:
a) providing a sample comprising blood cells from said human subject;
b) adding to the sample a reagent or reagents for detecting intracellular calcium in said sample;
c) measuring a baseline level of an intracellular calcium signal in said sample;
d) activating a PIEZO1 receptor in said sample to induce calcium influx;
e) measuring a level of an intracellular calcium signal in response of the activation of PIEZO1 in the sample;
f) calculating an increase in the level of the intracellular calcium signal from step c) to step e), wherein the increase in the level of the intracellular calcium signal from step c) to step e) provides a parameter, which parameter is used to determine the risk of development or risk of presence of Alzheimer's disease in said human subject,
g) comparing the obtained parameter to a known reference value for a subject with little or no risk of having Alzheimer's disease and/or to a known reference value for a subject with Alzheimer's disease to determine.

In an embodiment a method for determining a risk of development or risk of presence of Alzheimer's disease in a human subject comprises the steps of:
a) providing a sample comprising red blood cells (RBCs) from said human subject;
b) adding to the sample a fluorochrome for detecting intracellular calcium in said sample;
c) measuring a baseline level of an intracellular calcium signal in said sample;
d) activating a PIEZO1 receptor by Yoda1, Jedi1, or Jedi2 in said sample to induce calcium influx;
e) measuring a level of an intracellular calcium signal in response of the activation of PIEZO1 in the sample;
f) calculating an increase in the level of the intracellular calcium signal from step c) to step e), wherein the increase in the level of the intracellular calcium signal from step c) to step e) provides a parameter, which parameter is used to determine the risk of development or risk of presence of Alzheimer's disease in said human subject.

In an embodiment a method for determining a risk of development or risk of presence of Alzheimer's disease in a human subject comprises the steps of
a) providing a sample comprising red blood cells (RBCs) from said human subject;
b) adding to the sample a fluorochrome for detecting intracellular calcium in said sample;
c) measuring a baseline level of an intracellular calcium signal in said sample;
d) activating a PIEZO1 receptor by Yoda1, Jedi1, or Jedi2 in said sample to induce calcium influx;
e) measuring a level of an intracellular calcium signal in response of the activation of PIEZO1 in the sample;
f) calculating an increase in the level of the intracellular calcium signal from step c) to step e), wherein the increase in the level of the intracellular calcium signal from step c) to step e) provides a parameter, which parameter is used to determine the risk of development or risk of presence of Alzheimer's disease in said human subject,
g) comparing the obtained parameter to a known reference value for a subject with little or no risk of having Alzheimer's disease and/or to a known reference value for a subject with Alzheimer's disease to determine.

In an embodiment a method for determining a risk of development or risk of presence of Alzheimer's disease in a human subject comprises the steps of:
a) providing a sample comprising red blood cells (RBCs) from said human subject;
b) washing the blood cells with physiological buffer and centrifugation;
c) adding to the sample a fluorochrome for detecting intracellular calcium in said sample;
d) measuring a baseline level of an intracellular calcium signal in said sample;
e) activating a PIEZO1 receptor by Yoda1, Jedi1, or Jedi2 in said sample to induce calcium influx;
f) measuring a level of an intracellular calcium signal in response of the activation of PIEZO1 in the sample;
g) calculating an increase in the level of the intracellular calcium signal from step d) to step f), wherein the increase in the level of the intracellular calcium signal from step d) to step f) provides a parameter, which parameter is used to determine the risk of development or risk of presence of Alzheimer's disease in said human subject,
h) comparing the obtained parameter to a known reference value for a subject with little or no risk of having Alzheimer's disease and/or to a known reference value for a subject with Alzheimer's disease to determine
   wherein the Alzheimer' disease is prodromal Alzheimer' disease or Alzheimer' disease with dementia.

In an embodiment a method for determining a presence or risk of Alzheimer's disease by analyzing activity of PIEZO1 receptor comprises the steps of
a) providing a sample of peripheral blood;
b) adding a fluorochrome into the sample;
c) measuring the baseline level of the sample;
d) adding a PIEZO1 agonist, preferably Yoda1, into the sample to obtain a sample with activated PIEZO1 receptors;
e) measuring the sample obtained in step d) with a fluorometer or a fluorescence activated cell sorter (FACS);
f) obtaining a signal or signals from the sample;
g) assessing the obtained signals from the sample relative to the baseline signal from the sample, wherein the elevated level of the activity of PIEZO1 is indicative of Alzheimer's disease.

In an embodiment a method for determining a presence or risk of Alzheimer's disease by analyzing activity of PIEZO1 receptor comprises the steps of
a) providing a sample of peripheral blood;
b) washing the blood cells with physiological buffer and centrifugation;
c) adding a fluorochrome into the sample;
d) measuring the baseline level of the sample;
e) adding a PIEZO1 agonist, preferably Yoda1, Jedi1, or Jedi2, into the sample to obtain a sample with activated PIEZO1 receptors;
f) measuring the sample obtained in step d) with a fluorometer or a fluorescence activated cell sorter (FACS);
g) obtaining a signal or signals from the sample;
h) assessing the obtained signals from the sample relative to the baseline signal from the sample, wherein the elevated level of the activity of PIEZO1 is indicative of Alzheimer's disease.

In an embodiment the method may comprise several steps including but not limited to:
1) Providing a sample from vein, artery, or fingertip,
2) Separation of the RBC fraction by centrifugation,
3) Washing the RBC fraction with physiological buffer and centrifugation,
4) Addition of the Ca²⁺ inducible fluorochrome such as Fluo4 am into the sample,
5) Measurement of the baseline fluorescence of the sample,
6) Addition of the PIEZO1 agonist such as Yoda1, Jedi1, or Jedi2 into the sample,
7) Measurement of the fluorescence,
8) Assessment of the result by comparing the signals between the sample and the baseline.

In an embodiment of the disclosure, the test method can be based on a use of a fluorescence activated cell sorter (FACS) to measure signals from the samples. In this method, the samples are fed into the FACS device from a test tube one by one, or from a compatible microtiter plate if such an option is available at the particular FACS device.

In another embodiment of the disclosure, the known amount of sample is distributed into the microtitration plate having one or more wells to enable screening of many samples in a short period of time. The result can be obtained with a fluorometer which can read one or multiple wells at the same time. This method allows automation of the sample handling and addition of the reagents.

The disclosure describes a kit for determining a risk of development of Alzheimer's disease or risk of presence of Alzheimer's disease, wherein the kit comprises a receptacle, an intracellular calcium indicator, a PIEZO1 activator, and optionally washing buffer.

Typically, the receptacle is a test tube or microtiter plate. An aliquot of a sample, such as an aliquot of blood sample, is added to a suitable receptacle.

The test tube may be a conventional test tube suitable for blood samples.

The microtiter plate may be a microtiter plate with one or more wells to enable high throughput analysis of samples. High throughput analysis may be carried out with a fluorometer.

A skilled person in the art can choose a suitable receptacle or receptacles, such as test tubes and/or microtiter plates.

In a further embodiment, a kit can comprise instructions for use. For example, the instructions may inform how to collect the sample, process the sample, conduct the measurement and analyze results.

In an embodiment the PIEZO1 activator contained in the kit is a PIEZO1 agonist. In a preferred embodiment the PIEZO1 agonist is Yoda 1, Jedi1, or Jedi2. In a more preferred embodiment the PIEZO1 agonist is Yoda1.

In an embodiment the intracellular calcium indicator contained in the kit is a fluorochrome. In a preferred embodiment the intracellular calcium indicator is a calcium reactive fluorochrome. The calcium reactive fluorochrome reacts with calcium and gives a signal measurable with a fluorometer. An example of a fluorochrome is Fluo4 am.

The methods, which are applicable to measure PIEZO1 gating activity by means of Ca²⁺ induced fluorescence include fluorescence activated cell sorter (FACS), and in a more advantageous embodiment, a high through-put screening (HTS) method using a microtitration plate with one or more wells. The HTS method enables to measure large number of samples in a short time with automation.

In an embodiment the kit comprises a washing buffer. In a preferred embodiment a skilled person can select a suitable washing buffer. For example, the washing buffer may be selected from the group consisting of phosphate-buffered saline (PBS and Hank's Balanced Salt Solution (HBSS).

In a preferred embodiment the kit comprises further components. For example, the kit may comprise reagents for use in the measurement of the intracellular calcium level. The kit may comprise medium or media wherein the blood cells, such as red blood cells (RBCs) are resuspended. An example of a suitable medium is RPMI medium supplemented with 10% FBS.

In an embodiment the kit for determining a risk of development or risk of presence of Alzheimer's disease comprises a receptacle, a fluorochrome, a PIEZO1 agonist, and optionally washing buffer. In an embodiment the test kit for determining a risk of development or risk of presence of Alzheimer's disease comprises a receptacle, a fluorochrome, a PIEZO1 agonist, and washing buffer.

In an embodiment the test kit for determining a risk of development or risk of presence of Alzheimer's disease comprises a receptacle, a fluorochrome, Yoda1, and optionally washing buffer. In an embodiment the test kit for determining a risk of development or risk of presence of Alzheimer's disease comprises a receptacle, a fluorochrome, Yoda1, and washing buffer.

In an embodiment the test kit for determining a risk of development or risk of presence of Alzheimer's disease comprises a receptacle, a fluorochrome, Jedi1, and optionally washing buffer. In an embodiment the test kit for determining a risk of development or risk of presence of Alzheimer's disease comprises a receptacle, a fluorochrome, Jedi1, and washing buffer.

In an embodiment the test kit for determining a risk of development or risk of presence of Alzheimer's disease comprises a receptacle, a fluorochrome, Jedi2, and optionally washing buffer. In an embodiment the test kit for determining a risk of development or risk of presence of Alzheimer's disease comprises a receptacle, a fluorochrome, Jedi2, and washing buffer.

Further, the disclosure describes an in vitro use of a PIEZO1 receptor as a biomarker for determining a risk of development of Alzheimer's disease or risk of presence of Alzheimer's disease in a human subject. In an embodiment the use is in vitro use. Alzheimer's disease may be prodromal Alzheimer's disease or Alzheimer's disease with dementia.

In an embodiment the PIEZO1 receptor is activated, said activation resulting in an increased level of an intracellular calcium signal compared to a baseline level of an intracellular calcium signal thereby providing a parameter, which parameter is used to determine the risk of development of Alzheimer's disease or risk of presence of Alzheimer's disease in said human subject by comparing the obtained parameter to a known reference value for a subject with little or no risk of having Alzheimer's disease and/or to a known reference value for a subject having Alzheimer's disease.

In an embodiment an increase in a value of a parameter obtained from an increased level of an intracellular calcium signal compared to a baseline level of an intracellular calcium signal indicates an increased risk of development of Alzheimer's disease or increased risk of presence of Alzheimer's disease in said human subject. a baseline level of an intracellular calcium signal in said sample.

In an embodiment an increased level of an intracellular calcium signal in response of an activation of the PIEZO1 receptor provides a parameter, which parameter is used to determine the risk of development or risk of presence of Alzheimer's disease in said human subject by comparing the obtained parameter to a known reference value for a subject with little or no risk of having Alzheimer's disease and/or to a known reference value for a subject with Alzheimer's disease to determine.

In an embodiment the use of a PIEZO1 receptor as a biomarker for determining a risk of development or risk of presence of Alzheimer's disease in a human subject is characterized by an increased level of an intracellular calcium signal in response of an activation of the PIEZO1 receptor provides a parameter, which parameter is used to determine the risk of development or risk of presence of Alzheimer's disease in said human subject by comparing the obtained parameter to a known reference value for a subject with little or no risk of having Alzheimer's disease and/or to a known reference value for a subject with Alzheimer's disease to determine.

In an embodiment the in vitro use of a PIEZO1 receptor as a biomarker for determining a risk of development or risk of presence of Alzheimer's disease in a human subject is characterized by an increased level of an intracellular calcium signal in response of an activation of the PIEZO1 receptor provides a parameter, which parameter is used to determine the risk of development or risk of presence of Alzheimer's disease in said human subject by comparing the obtained parameter to a known reference value for a subject with little or no risk of having Alzheimer's disease and/or to a known reference value for a subject with Alzheimer's disease to determine.

In an embodiment the biomarker of the present disclosure is the in vitro use of a PIEZO1 receptor for determining a risk of development or risk of presence of Alzheimer's disease in a human subject, wherein an increased level of an intracellular calcium signal in response of an activation of the PIEZO1 receptor provides a parameter, which parameter is used to determine the risk of development or risk of presence of Alzheimer's disease in said human subject by comparing the obtained parameter to a known reference value for a subject with little or no risk of having Alzheimer's disease and/or comparing the obtained parameter to a known reference value for a subject with Alzheimer's disease to determine.

In an embodiment read-out from the human subject's sample is analyzed against a probability distribution, assigning for each read-out a probability of being identified as healthy, a probability for being identified as having prodromal Alzheimer's disease and a probability for being identified having Alzheimer's disease with dementia, wherein the probability distributions are based on previously analyzed cohorts of donors for each respective subject group.

Further, the disclosure describes an in vitro use of determining intracellular calcium level in response to the activation of PIEZO1 as a biomarker, wherein the biomarker is used for determining a risk for development of Alzheimer's disease or risk for presence of Alzheimer's disease in a human subject.

In an embodiment a PIEZO1 receptor is activated, said activation resulting in an increased level of an intracellular calcium signal compared to a baseline level of an intracellular calcium signal thereby providing a parameter, which parameter is used to determine the risk of development of Alzheimer's disease or risk of presence of Alzheimer's disease in said human subject by comparing the obtained parameter to a known reference value for a subject with little or no risk of having Alzheimer's disease and/or to a known reference value for a subject with Alzheimer's disease.

In an embodiment an increase in a value of a parameter obtained from an increased level of an intracellular calcium signal compared to a baseline level of an intracellular calcium signal indicates an increased risk of development of Alzheimer's disease or increased risk of presence of Alzheimer's disease in said human subject.

An aspect of the present disclosure relates to an AD biomarker and its use thereof. As used herein, the AD biomarker refers to a biomolecule, which can be used to assess the subjects who will be most likely to develop an AD. Analysis of the biomarker is especially useful for early detection of AD. As used herein, the biomarker refers to a PIEZO1 receptor found in many different cells in human body, and more specifically, the biomarker refers to the activity of the PIEZO1 receptor in RBCs as a function of its specific agonists such as Yoda1. It regulates many vital functions in human and its dysfunction is shown to be related to the Aβ pathology and neuroinflammation in brain. As used herein, “in blood” means that the biomarker exists in RBCs and can be detected in blood specimen.

In an embodiment the kit is used for differentiating healthy subjects and subjects with Alzheimer's disease. In another embodiment the kit is used for differentiating healthy subjects and subjects with mild cognitive impairment. In an embodiment the kit is used for differentiating healthy subjects and subjects with prodromal Alzheimer's disease.

In an embodiment the kit is used for differentiating healthy subjects and subjects with AD.

The present disclosure provides a biomarker useful in detection, diagnosis, differentiation, and prognosis of cognition impairment especially related to the Alzheimer's disease. The biomarker is a PIEZO1 receptor and its activity when agonized with molecules, which intervene the gating function of the receptor. In Alzheimer's disease or in mild cognitive impairment, gating function of the PIEZO1 receptor is changed when compared to the subjects having no symptoms of impaired cognition. The difference in the gating function is measurable with the method disclosed. Even though, the MCI and AD has been regarded as a dysfunction of the brain, the effect is seen also in peripheral cell such as red blood cells. This makes it possible to develop minimum invasive methods to analyze the state of the MCI and AD. It is previously known that red blood cells express PIEZO1 receptors, but the present invention is based on a new scientific discovery according to which alteration of the gating function as a response to the PIEZO1 agonists in red blood cells relates to development of Alzheimer's disease. The method disclosed is not limited to the examples or embodiments shown in the text since various modifications can be achieved by a skilled person in the art. Industrial applicability of the present invention relates to the diagnosis of the Alzheimer's disease, especially by differentiating the early progressive subjects from the healthy. This can speed up initiation of the treatments, which can slow down progression and onset of the disease.

The present invention is not limited to these embodiments since the biomarker can be analyzed manually by using a test tube and a fluorometer including but not excluding the typical steps of the method.

It is apparent to a person skilled in the art that as technology advances, the basic idea of the invention can be implemented in various ways. The invention and its embodiments are therefore not restricted to the examples below, but they may vary within the scope of the claims.

### EXAMPLES

### Patient Cohorts Analyzed

The results presented in the Examples 1 and 2, are based on the patient cohorts recruited under the ethical licenses of health district of Northern Savo, permission 536/13.02.00/2017 and Kuopio University Hospital permission 482/2017. The donors were >60 years old and classified based on the Clinical Dementia Rating score (CDR), with the healthy controls identified as CDR=0, prodromal AD as CDR=0.5 and, AD with mild dementia as CDR=1. The classification was refined by further cognitive testing (CERAD). Subsequently, controls without previously known cognitive decline were re-categorised to prodromal AD group if they showed cognitive problems in CERAD and to AD dementia group if screening interviews also revealed deterioration in daily functions. Demographic interview was carried out and medical records were checked to exclude the persons with other memory disorder than AD from the study. Also, structural MRI assessment was performed to exclude patients with cerebrovascular changes or other conditions known to impact cognition.

### Example 1

Analysis of the Samples Collected from the Healthy, MCI, and Alzheimer's Patients Using the Test Tube and FACS to Read the Result.

The venous blood samples from healthy subjects, patients with mild cognitive impairment (MCI), and patients with Alzheimer's disease (AD) were collected into BD Vacutainer® blood collection tubes containing the K3-EDTA anti-coagulant. 150 μl of whole blood was taken for further processing, washed two times with 10 ml of phosphate-buffered saline (PBS) (5 mins, 300 g, RT (=about 20-25° C.) to remove plasma. After the second wash, the supernatant was removed and 50 μl of red blood cells (RBCs) were transferred to clean falcon tube and resuspended in 200 μl PBS (250 μl total volume of cell suspension). 250 μl of Fluo4 am AM (Thermo Fisher Scientific) fluorescent dye was added and cells were incubated for 30 minutes at 37° C. in dark.

After the incubation, cell suspension was washed twice with 5 ml of PBS, followed by one wash with HBSS (5 mins, 300 g, RT). Supernatant was carefully removed with pipette and 50 μl pellet was resuspended in 450 μl of Hank's Balanced Salt Solution (HBSS) to form 10% cell suspension. 10 μl of 10% cell suspension was transferred to 390 μl of RPMI/FBS (10:1) (400 μl final volume). This cell suspension was further used for flow cytometry time-lapse assessment using CytoFLEX S flow cytometer (Beckman Coulter, USA).

At the start of the recording, at t=0 seconds, baseline fluorescence was recorded for 20 seconds. At t=20 seconds, Yoda1 in DMSO was applied to the sample at the final concentration of 5 or 10 μM. At t=4 minutes and 20 seconds, ionomycin, serving as the positive control, was applied at the final concentration 10 μM. Recording was finished at t=5 minutes 30 seconds.

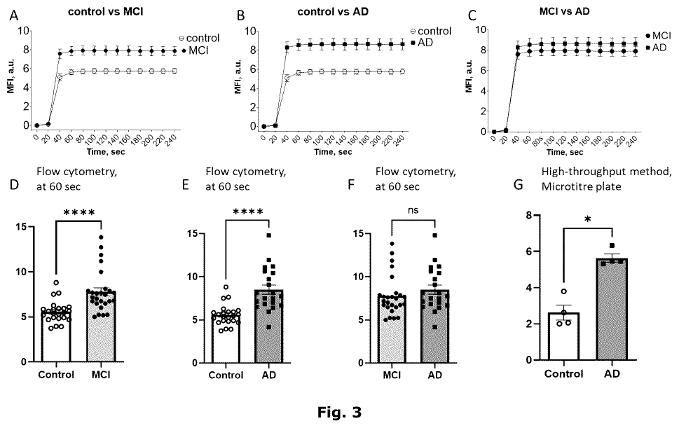

FIGS. 3A-3C plot the mean values of Ca²⁺ signal for each group with 10 μM Yoda1 spanning from BL (t=0 seconds) to t=4 minutes, while the FIGS. 3D-3F show the measurements of individual samples at t=60 seconds.

All flow cytometric data were analyzed using CytExpert Software v 2.4 (Beckman Coulter Inc., CA, USA). Yoda1-induced Ca flux was measured by using Fluo4 am median fluorescence intensity (MFI) of single cells after Yoda1 application at different time points—baseline (before application of Yoda1 at 10 μM concentrations), and every 20 sec of the recording.

### Statistical Analysis

Data were analyzed using GraphPad Prism Software (CA, USA). Statistical analysis was performed using Mann-Whitney test, two-tailed. The data are presented as mean±SEM.

### Results

To compare the gating properties of PIEZO1 in red blood cells of healthy donors, MCI patient and AD patients, Ca influx in the respective sample groups upon exposure to specific PIEZO1 agonist Yoda1 was recorded and quantified.

In response to Yoda1 application at 10 μM concentrations we observed rapid increase in Fluo4 am MFI levels, indicating the Ca²⁺ influx into the red blood cells (RBCs) through PIEZO1 ion channels. Maximum levels were reached by 60 s and remained at the similar levels till the end of 4 min recording of Yoda1 response. (FIGS. 3A-3C).

We found that Yoda1-induced response in control subjects was lower compared to MCI and AD patients (FIGS. 3A-3G). When stimulating RBCs with 10 μM of Yoda1, at 60 s of recording the normalized MFI levels reached 5.64±0.27 in control, 7.9±0.48 in MCI and 8.57±0.6 in AD groups (mean±SEM, FIGS. 3D-3F). The difference was increasing with disease progression and the biggest difference was observed between control and AD group (FIG. 3F). The differences were statistically significant, with p<0.0001, between healthy and MCI and between healthy and AD, but not significant (p>0.05) between MCI and AD. Healthy n=23, MCI n=26 and AD n=21.

### Example 2

Analysis of the Samples Collected from the Healthy and Alzheimer's Patients Using the Microtiter Plate Format and a Fluorometer to Read the Result.

300 μL of blood from the healthy persons and AD patients was collected into the blood sampling tubes with sodium citrate to prevent coagulation. The samples were washed twice with PBS buffer (Ca²⁺ and Mg²⁺ free) by centrifugation at 300 G for 5 minutes. 150 μL of the red blood cell (RBC) pellet was divided into two parts, 50 μL for fluorescent staining and 50 μL for the negative control. 100 μL of the RBC were resuspended in 500 μL of PBS, to which Fluo4 am was added and incubated in the dark for 30 min at 37° C. The RBC suspension was then washed twice with PBS to remove the dye, which was not taken into the cells, and centrifuged for 5 min at 300 G at RT (20-25° C.). The RBC suspension was washed with HBSS (with Ca²⁺ and Mg²⁺) followed by centrifugation for 5 min at 300 G at RT. 100 μL of the RBC pellet loaded with Fluo4 am was resuspended in 15 mL of HBSS (with Ca²⁺ and Mg²⁺). The control included 50 μL of the RBC resuspended in 7.5 mL of HBSS (with Ca²⁺ and Mg²⁺). The samples were distributed into the microtitration plate (96-wells, 180 μL per well), after which the ground level of fluorescence (485 nm/535 nm, 1.0 s, Victor Wallac) was measured immediately. 20 μL of DMSO (for vehicle control) or 20 μL of 5 μM Yoda1 (for PIEZO1 activity) was added into the wells, and the fluorescence was measured immediately. The fluorescence signal of the sample having no Fluo4 am was subtracted from the fluorescence signals of the vehicle and Yoda1 samples treated with Fluo4 am. The increase in fluorescence upon addition of Yoda1 was normalized to the signal measured before addition of Yoda1. The normalized increase fluorescence was 2.62±0.42 for healthy and 5.64±0.23 for AD (mean #SEM) (FIG. 3G). As shown, the PIEZO1 response to the Yoda1 agonist was greater in the AD samples compare to the healthy. Even though, the number of observations was low (4) in both groups, the difference was statistically significant, with p<0.05, and in line with the results in Example 1.

### REFERENCES

Abner E L., et al. Outcomes after diagnosis of mild cognitive impairment in a large autopsy series. Ann Neurol 2017; 81:549-559. doi: 10.1002/ana.24903.
Ashton N J., et al. Update on biomarkers for amyloid pathology in Alzheimer's disease. Biomarkers Med. 2018; 12 (7): 799-812.
Cahalan, S. M.; Lukacs, V.; Ranade, S. S.; Chien, S.; Bandell, M.; Patapoutian, A. Piezo1 Links Mechanical Forces to Red Blood Cell Volume. Elife 2015, 4, doi: 10.7554/eLife.07370
Coste et al., Piezo1 and Piezo2 Are Essential Components of Distinct Mechanically Activated Cation Channels. Science. 2010, 330, 55-60, doi: 10.1126/science.1193270.
Cummings J L., et al. Alzheimer's disease drug development pipeline: few candidates, frequent failures. Alzheimers. Res Ther 2014; 6:37. doi: 10.1186/alzrt269.
Fang, X.-Z.; Zhou, T.; Xu, J.-Q.; Wang, Y.-X.; Sun, M.-M.; He, Y.-J.; Pan, S.-W.; Xiong, W.; Peng, Z.-K.; Gao, X.-H.; et al. Structure, Kinetic Properties and Biological Function of Mechanosensitive Piezo Channels. Cell Biosci. 2021, 11, 13, doi: 10.1186/s13578-020-00522-z.
Grothe M J., et al. Associations of fully automated CFS and novel plasma biomarkers with h Alzheimer's disease neuropathology at autopsy. Neurology. 2021; 97: e1229-e1242. doi: 10.1212/WNL.0000000000012513.
Jack C R Jr., et al. NIA-AA Research Framework: toward a biological definition of Alzheimer's disease. Alzheimers Demen. 2018; 14 (4): 535-562
Jiang, Y.; Yang, X.; Jiang, J.; Xiao, B. Structural Designs and Mechanogating Mechanisms of the Mechanosensitive Piezo Channels. Trends Biochem. Sci. 2021, 46, 472-488, doi: https://doi.org/10.1016/j.tibs.2021.01.008.
Maneshi M M., et al. Enantiomeric Aβ peptides inhibit the fluid shear stress response of PIEZO1. Scientific Reports (2018) 8:14267, DOI: 10.1038/s41598-018-32572-2.
Mattsson N., et al. CSF biomarkers and incipient Alzheimer disease in patients with mild cognitive impairment. JAMA. 2009; 302:385-393. doi: 10.1001/jama.
O'Boyle, N. M., Banck, M., James, C. A. et al. Open Babel: An open chemical toolbox. J Cheminform 3, 33 (2011). https://doi.org/10.1186/1758-2946-3-33
Petersen R C., et al. Current concepts in mild cognitive impairment. Arch Neurol 2001; 58:1985-1992. doi: 10.1001/archneur.58.12.1985
Scholl M., et al. Biomarkers for tau pathology. Mol Cell Neurosci. 2019; 97:18-33.
Sitnikova V., et al. Microglial beta-amyloid clearance is driven by PIEZO1 channels. Submitted (2021).
Syeda, R.; Xu, J.; Dubin, A. E.; Coste, B.; Mathur, J.; Huynh, T.; Matzen, J.; Lao, J.; Tully, D. C.; Engels, I. H.; et al. Chemical Activation of the Mechanotransduction Channel Piezo1. Elife 2015, 4, doi: 10.7554/eLife.07369.
Wang, S.; Chennupati, R.; Kaur, H.; Iring, A.; Wettschureck, N.; Offermanns, S. Endothelial Cation Channel PIEZO1 Controls Blood Pressure by Mediating Flow-Induced ATP Release. J. Clin. Invest. 2016, 126, 4527-4536, doi: 10.1172/JCI87343.
Wang, Y.; Chi, S.; Guo, H.; Li, G.; Wang, L.; Zhao, Q.; Rao, Y.; Zu, L.; He, W.; Xiao, B. A Lever-like Transduction Pathway for Long-Distance Chemical- and Mechano-Gating of the Mechanosensitive Piezo1 Channel. Nat. Commun. 2018, 9, 1300, doi: 10.1038/s41467-018-03570-9.
Ward, A.; Tardiff, S.; Dye, C.; Arrighi, H. M. Rate of Conversion from Prodromal Alzheimer's Disease to Alzheimer's Dementia: A Systematic Review of the Literature. Dement. Geriatr. Cogn. Dis. Extra 2013, 3, 320-332, doi:10.1159/000354370.
Wei H C., et al. Amyloid and tau positive mild cognitive impairment: clinical and biomarker characteristics of dementia progression. Chinese Medical Journal. 2021; 134 (14).

1. A method for determining a risk of development or risk of presence of Alzheimer's disease in a human subject, the method comprising:
a) providing an in vitro sample comprising blood cells from said human subject;
b) adding to the sample a reagent or reagents for detecting intracellular calcium in said sample;
c) measuring a baseline level of an intracellular calcium signal in said sample;
d) activating a PIEZO1 receptor present in said sample to induce calcium influx;
e) measuring a level of an intracellular calcium signal in response of the activation of PIEZO1 in the sample; and
f) calculating an increase in the level of the intracellular calcium signal from step c) to step e),
wherein the increase in the level of the intracellular calcium signal from step c) to step e) provides a parameter, which parameter is used to determine the risk of development or risk of presence of Alzheimer's disease in said human subject,
wherein increase in a value of the parameter obtained in step f) indicates an increased risk of development of Alzheimer's disease or increased risk of presence of Alzheimer's disease in said human subject.

2. The method according to claim 1, wherein the Alzheimer's disease is prodromal Alzheimer's disease or Alzheimer's disease with dementia.

3. The method according to claim 1 wherein the method further comprises washing the blood cells with a physiological buffer and centrifugation after step a).

4. The method according to claim 1, wherein the method further comprises comparing the obtained parameter to a known reference value for a subject with little or no risk of having Alzheimer's disease and/or to a known reference value for a subject with Alzheimer's disease to determine a risk of development or risk of presence of Alzheimer's disease in a human subject.

5. The method according to claim 1, wherein the blood cells comprise red blood cells.

6. The method according to claim 1, wherein the reagent or reagents for detecting intracellular calcium comprises a fluorochrome.

7. The method according to claim 1, wherein the PIEZO1 receptor is activated by a PIEZO1 agonist.

8. The method according to claim 1, wherein the method is an in vitro method.

9. A kit for determining a risk of development or risk of presence of Alzheimer's disease according to the method of claim 1, wherein the kit comprises, a receptacle, an intracellular calcium indicator, a PIEZO1 activator, and optionally washing buffer.

10. The kit according to claim 9, wherein the PIEZO1 activator is a PIEZO1 agonist.

11. The kit according to claim 9, wherein the receptacle is a test tube or microtiter plate.

12. The kit according to claim 1, wherein the intracellular calcium indicator is a fluorochrome.

13. The kit according to claim 9, wherein the washing buffer is selected from the group consisting of phosphate-buffered saline (PBS) and Hank's Balanced Salt Solution (HBSS).

14. An in vitro use of a PIEZO1 receptor as a biomarker, wherein the biomarker is used for determining a risk for development of Alzheimer's disease or risk for presence of Alzheimer's disease in a human subject.

15. The use according to claim 14, wherein the Alzheimer's disease is prodromal Alzheimer's disease or Alzheimer's disease with dementia.

16. The use according to claim 14, wherein the PIEZO1 receptor is activated, said activation resulting in an increased level of an intracellular calcium signal compared to a baseline level of an intracellular calcium signal thereby providing a parameter, which parameter is used to determine the risk of development of Alzheimer's disease or risk of presence of Alzheimer's disease in said human subject by comparing the obtained parameter to a known reference value for a subject with little or no risk of having Alzheimer's disease and/or to a known reference value for a subject having Alzheimer's disease.

17. The use according to claim 14, wherein an increase in a value of a parameter obtained from an increased level of an intracellular calcium signal compared to a baseline level of an intracellular calcium signal in said sample indicates an increased risk of development of Alzheimer's disease or increased risk of presence of Alzheimer's disease in said human subject.

18. The use according to claim 14, wherein the PIEZO1 receptor is activated by a PIEZO1 agonist.

19. An in vitro use of determining intracellular calcium level in response to the activation of PIEZO1 as a biomarker, wherein the biomarker is used for determining a risk for development of Alzheimer's disease or risk for presence of Alzheimer's disease in a human subject.

20. The use according to claim 19, wherein the Alzheimer's disease is prodromal Alzheimer's disease or Alzheimer's disease with dementia.

21. The use according to claim 19, wherein a PIEZO1 receptor is activated, said activation resulting in an increased level of an intracellular calcium signal compared to a baseline level of an intracellular calcium signal thereby providing a parameter, which parameter is used to determine the risk of development of Alzheimer's disease or risk of presence of Alzheimer's disease in said human subject by comparing the obtained parameter to a known reference value for a subject with little or no risk of having Alzheimer's disease and/or to a known reference value for a subject with Alzheimer's disease.

22. The use according to claim 19, wherein an increase in a value of a parameter obtained from an increased level of an intracellular calcium signal compared to a baseline level of an intracellular calcium signal indicates an increased risk of development of Alzheimer's disease or increased risk of presence of Alzheimer's disease in said human subject.

23. The use according to claim 19, wherein the PIEZO1 receptor is activated by a PIEZO1 agonist.
